(19) United States
(12) Patent Application Publication
(10) Pub. No.: US 2010/0022590 A1
(43) Pub. Date: Jan. 28, 2010

(54) Novel compounds

(75) Inventors: Styrbjörn Byström, Taby (SE); Martin Haraldsson, Taby (SE); Lars Johansson, Bromma (SE); Jan Tejbrant, Bandhagen (SE)

Correspondence Address: EDWARDS ANGELL PALMER & DODGE LLP, P.O. BOX 55874, BOSTON, MA, 02205, US

(73) Assignee: BIOVITRUM AB (publ.), Stockholm (SE)

(21) Appl. No.: 12/321,096

(22) Filed: Jan. 15, 2009

(30) Foreign Application Priority Data
Jan. 17, 2008 (SE) 0800108-3

(51) Int. Cl. A61K 31/445; C07D 413/06

(52) U.S. Cl. 514/326; 546/209

(57) ABSTRACT

The present invention relates to novel isoxazole compounds of formula (I),

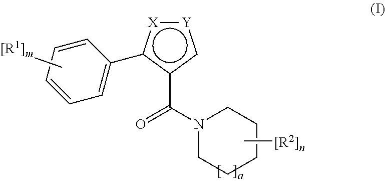

and pharmaceutically acceptable salts, solvates, hydrates, geometrical isomers, tautomers, optical isomers or N-oxides thereof, which are modulators of 11β-hydroxysteroid dehydrogenase type 1 (11β-HSD1). The invention also relates to pharmaceutical compositions comprising these compounds and to the use of these compounds in the preparation of a medicament for the treatment of glaucoma.

### FIELD OF THE INVENTION

The present invention relates to novel isoxazole compounds of formula (I), which are modulators of 11β-hydroxysteroid dehydrogenase type 1 (11β-HSD1) and can be used for the treatment of medical conditions connected with 11β-HSD1 activity. The invention also relates to pharmaceutical compositions comprising these compounds, to the use of these compounds in the preparation of a medicament for the treatment of glaucoma, as well as to processes for the preparation of these compounds.

### BACKGROUND ART

Glaucoma, a heterogeneous group of diseases of which primary open-angle glaucoma (POAG) is the most prevalent, is a leading cause of irreversible visual loss responsible for 14% of global blindness. It is characterized by an optic neuropathy with corresponding visual field loss, associated with a range of risk factors including elevated intraocular pressure (IOP), which is possible to treat and control. The IOP is regulated by a fine balance between production of aqueous humour (AH) by the ciliary epithelium, and drainage via the trabecular meshwork (TM), the canal of Schlemm and uveoscleral outflow routes. This process may be regulated by corticosteroids, since approximately one third of the normal population treated with topical corticosteroids develop a moderate increase of the IOP, while virtually all patients with POAG develop increased IOP after topical corticosteroid therapy [Armaly, Arch. Opthalmol. 1963, 70, 483-491; Armaly, Arch. Opthalmol. 1963, 70, 492-499; Becker, Invest. Opthalmol. 1965, 4, 198-205; Armaly, Arch. Opthalmol. 1967, 77, 747-751]. In addition, patients suffering from Cushing's syndrome develop an increased IOP [Sayegh et al., Ophthalmic Res. 1975, 7, 390-394]. Occupancy and activation of steroid hormone receptors is regulated by hydroxysteroid dehydrogenases (HSDs), which convert steroid hormones into their inactive metabolites [for a recent review see Nobel et al., Eur. J. Biochem. 2001, 268, 4113-4125]. Numerous classes of HSDs exist, of which the 11β-hydroxysteroid dehydrogenases (11β-HSDs) catalyze the interconversion of active glucocorticoids (such as cortisol and corticosterone), and their inert forms (such as cortisone and 11-dehydrocorticosterone). The isoform 11β-hydroxysteroid dehydrogenase type 1 (11β-HSD1), which activates cortisone to cortisol, is expressed among others in liver, adipose tissue, brain, lung and other glucocorticoid tissue and is a potential target for therapy directed at numerous disorders that may be ameliorated by reduction of glucocorticoids action, such as diabetes, obesity and age-related cognitive dysfunction [Seckl et al., Endocrinology 2001, 142, 1371-1376].

Interestingly, in a pilot uncontrolled study in healthy volunteers, the nonselective 11β-HSD1/11β-HSD2 inhibitor carbenoxolone (CBX) was shown to reduce intraocular pressure (IOP) with 20% upon oral administration [Rauz et al., Invest. Opthalmol. Vis. Sci. 2001, 42, 2037-2042]. Similarly, in a placebo-controlled study of patients with ocular hypertension, orally administered CBX induced 10% IOP reduction [Rauz et al., Q. J. Med. 2003, 96, 481-490]. Tissue expression studies have shown the presence of 11β-HSD1, but not 11β-HSD2 in human ciliary epithelial cells. This predominant 11β-HSD1 expression in ocular tissues was supported by excessive levels of cortisol versus cortisone in AH of both healthy subjects and patients, while the reverse was observed in urine, is reflecting 11β-HSD2 activity in the kidney. Taken together, the results suggest that selective inhibition of 11β-HSD1 in the eye may be a valid approach for reducing an already elevated IOP, and hence treating glaucoma [U.S. Pat. No. 6,548,053; see also Walker et al., poster P3-698 at the Endocrine Society Meeting Jun. 12-15, 1999, San Diego].

Topical application to the eye is the preferred route for pharmacological intervention of ocular diseases, since this results in high concentrations of the active compound at the desired site of action, while at the same time reducing the risk for systemic side effects. Aqueous solutions are commonly accepted as the preferred formulation for glaucoma drugs.

Several substituted isoxazole compounds are known from prior art. WO 01/29015 describes isoxazole derivatives with enhanced selectivity for the α₁ₐ adrenergic receptor for use in the treatment of obstructive syndromes of the lower urinary tract. WO 2007/114124 describes substituted isoxazoles derivatives as 11β-HSD1 inhibitors for the treatment of obesity.

However, it has not previously been shown that substituted isoxazole compounds are suitable for topical application to the eye for the treatment of glaucoma.

### DISCLOSURE OF THE INVENTION

It has surprisingly been found that the isoxazole compounds of the formula (I), which are potent and selective 11β-HSD1 inhibitors, have physicochemical properties which make them particularly suitable for topical application to the eye for the treatment of glaucoma.

In a first aspect, the invention relates to a compound of formula (I)

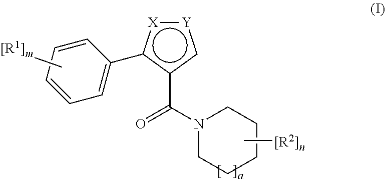

or a pharmaceutically acceptable salt, solvate, hydrate, geometrical isomer, tautomer, optical isomer or N-oxide thereof, wherein:
X—Y represents N—O or O—N;
R¹ is independently selected from the group consisting of halogen, cyano, CF₃, OCF₃, C₁₋₄-alkyl, hydroxy-C₁₋₄-alkyl, C₁₋₄-alkoxy-C₁₋₄-alkyl and C₁₋₄-alkoxy;
or two substituents R¹, together with the carbon atoms they are attached to, form a 5- or 6-membered aromatic or non-aromatic ring, which optionally contains one or more heteroatoms selected from O and N, and which ring is optionally substituted with one or more substituents selected from the group consisting of halogen, cyano, CF₃, OCF₃, C₁₋₄-alkyl, hydroxy-C₁₋₄-alkyl, C₁₋₄-alkoxy-C₁₋₄-alkyl and C₁₋₄-alkoxy;
R² is independently selected from the group consisting of C₁₋₈-alkyl, hydroxy-C₁₋₈-alkyl, C₁₋₈-alkoxy-C₁₋₈-alkyl, C₁₋₈-alkoxy, hydroxy-C₁₋₈-alkoxy, C₁₋₈-alkoxy-C₁₋₈-alkoxy, C₃₋₈-cycloalkyl, hydroxy-C₃₋₈-cycloalkyl, C₁₋₈-alkoxy-C₃₋₈-cycloalkyl, C₃₋₈-cycloalkyloxy, hydroxy-C₃₋₈-cycloalkyloxy and C₁₋₈-alkoxy-C₃₋₈-cycloalkyloxy;
a is 0, 1 or 2; and
m and n are each independently 0, 1 or 2;
with the proviso that the compound is not selected from the group consisting of:
1-[(5-phenyl-4-isoxazolyl)carbonyl]-piperidine;
5-phenyl-4-(pyrrolidin-1-ylcarbonyl)isoxazole;
5-(4-fluorophenyl)-4-(pyrrolidin-1-ylcarbonyl)isoxazole;
5-(4-chlorophenyl)-4-(pyrrolidin-1-ylcarbonyl)isoxazole;
5-(4-bromophenyl)-4-(pyrrolidin-1-ylcarbonyl)isoxazole;
5-(4-methylphenyl)-4-(pyrrolidin-1-ylcarbonyl)isoxazole;
5-(4-methoxyphenyl)-4-(pyrrolidin-1-ylcarbonyl)isoxazole;
4-{[2-(methoxymethyl)pyrrolidin-1-yl]carbonyl}-5-(4-methylphenyl)isoxazole;
4-{[(2S)-2-(methoxymethyl)pyrrolidin-1-yl]carbonyl}-5-(4-methylphenyl)isoxazole;
4-{[(2R)-2-(methoxymethyl)pyrrolidin-1-yl]carbonyl}-5-(4-methylphenyl)isoxazole;
5-(2-chlorophenyl)-4-{[2-(methoxymethyl)pyrrolidin-1-yl]carbonyl}isoxazole;
5-(2-chlorophenyl)-4-{[(2S)-2-(methoxymethyl)pyrrolidin-1-yl]carbonyl}isoxazole;
5-(2-chlorophenyl)-4-{[(2R)-2-(methoxymethyl)pyrrolidin-1-yl]carbonyl}isoxazole;
4-{[(2S)-2-(methoxymethyl)pyrrolidin-1-yl]carbonyl}-5-[4-(trifluoromethyl)phenyl]-isoxazole;
4-{[(2R)-2-(methoxymethyl)pyrrolidin-1-yl]carbonyl}-5-[4-(trifluoromethyl)phenyl]-isoxazole;
5-(4-chlorophenyl)-4-{[2-(isopropyl)pyrrolidin-1-yl]carbonyl}isoxazole;
2-(1-{[5-(4-methylphenyl)isoxazol-4-yl]carbonyl}piperidin-3-yl)propan-2-ol;
2-(1-{[5-(2-chlorophenyl)isoxazol-4-yl]carbonyl}piperidin-3-yl)propan-2-ol;
2-[1-({5-[4-(trifluoromethyl)phenyl]isoxazol-4-yl}carbonyl)piperidin-3-yl]propan-2-ol;
2-((3R)-1-{[3-(4-methylphenyl)isoxazol-4-yl]carbonyl}piperidin-3-yl)propan-2-ol;
2-((3R)-1-{[5-(4-methylphenyl)isoxazol-4-yl]carbonyl}piperidin-3-yl)propan-2-ol;
2-((3R)-1-{[3-(4-methoxyphenyl)isoxazol-4-yl]carbonyl}piperidin-3-yl)propan-2-ol;
2-((3R)-1-{[3-(3-chloro-4-methoxyphenyl)isoxazol-4-yl]carbonyl}piperidin-3-yl)-propan-2-ol;
3-(1-{[5-(4-methylphenyl)isoxazol-4-yl]carbonyl}piperidin-3-yl)pentan-3-ol;
3-(1-{[3-(4-methylphenyl)isoxazol-4-yl]carbonyl}piperidin-3-yl)pentan-3-ol;
3,5-dimethyl-1-{[5-(4-methylphenyl)isoxazol-4-yl]carbonyl}piperidine;
1-{[5-(2-chlorophenyl)isoxazol-4-yl]carbonyl}-3,5-dimethylpiperidine;
3,5-dimethyl-1-({5-[4-(trifluoromethyl)phenyl]isoxazol-4-yl}carbonyl)piperidine;
5-ethyl-2-methyl-1-{[5-(4-methylphenyl)isoxazol-4-yl]carbonyl}piperidine;
1-{[5-(2-chlorophenyl)isoxazol-4-yl]carbonyl}-5-ethyl-2-methylpiperidine; and
5-ethyl-2-methyl-1-({5-[4-(trifluoromethyl)phenyl]isoxazol-4-yl}carbonyl)piperidine.

In a preferred embodiment of the invention, R¹ is halogen or C₁₋₄-alkyl, or two substituents R¹, together with the carbon atoms they are attached to, form a 5- or 6-membered ring. In a most preferred embodiment, R¹ is F, Cl or methyl, or two substituents R¹, together with the carbon atoms they are attached to, form a 6-membered aromatic ring.

In another preferred embodiment, the heterocyclic ring bearing the substituent(s) R² is a piperidine ring. Therefore, a is preferably 1.

In yet another preferred embodiment, the piperidine ring is substituted with one substituent R². Therefore, n is preferably 1.

In yet another preferred embodiment, R² is hydroxy-C₁₋₈-alkyl or C₁₋₈-alkoxy-C₁₋₈-alkoxy. In a most preferred embodiment, R² is 1-hydroxyethyl, 2-hydroxyethyl or 1-hydroxy-1-methylethyl.

Specific preferred compounds according to the invention are those selected from the group consisting of:
2-(1-{[5-(2-methylphenyl)isoxazol-4-yl]carbonyl}piperidin-3-yl)propan-2-ol;
2-((3R)-1-{[5-(2-methylphenyl)isoxazol-4-yl]carbonyl}piperidin-3-yl)propan-2-ol;
2-((3S)-1-{[5-(2-methylphenyl)isoxazol-4-yl]carbonyl}piperidin-3-yl)propan-2-ol;
2-(1-{[5-(3-methylphenyl)isoxazol-4-yl]carbonyl}piperidin-3-yl)propan-2-ol;
2-((3S)-1-{[5-(4-methylphenyl)isoxazol-4-yl]carbonyl}piperidin-3-yl)propan-2-ol;
2-((3R)-1-{[5-(2-chlorophenyl)isoxazol-4-yl]carbonyl}piperidin-3-yl)propan-2-ol;
2-((3S)-1-{[5-(2-chlorophenyl)isoxazol-4-yl]carbonyl}piperidin-3-yl)propan-2-ol;
2-((3S)-1-{[5-(3-chlorophenyl)isoxazol-4-yl]carbonyl}piperidin-3-yl)propan-2-ol;
2-((3R)-1-{[5-(4-chlorophenyl)isoxazol-4-yl]carbonyl}piperidin-3-yl)propan-2-ol;
2-((3S)-1-{[5-(4-chlorophenyl)isoxazol-4-yl]carbonyl}piperidin-3-yl)propan-2-ol;
2-(1-{[5-(2-chlorophenyl)isoxazol-4-yl]carbonyl}piperidin-2-yl)ethanol;
2-(1-{[5-(3-chloro-2-methylphenyl)isoxazol-4-yl]carbonyl}piperidin-3-yl)propan-2-ol;
2-(1-{[5-(3,4-dimethylphenyl)isoxazol-4-yl]carbonyl}piperidin-3-yl)propan-2-ol;
2-((3R)-1-{[5-(3,4-dimethylphenyl)isoxazol-4-yl]carbonyl}piperidin-3-yl)propan-2-ol;
2-((3S)-1-{[5-(3,4-dimethylphenyl)isoxazol-4-yl]carbonyl}piperidin-3-yl)propan-2-ol;
2-((3R)-1-{[5-(2,4-dimethylphenyl)isoxazol-4-yl]carbonyl}piperidin-3-yl)propan-2-ol;
2-((3S)-1-{[5-(2,4-dimethylphenyl)isoxazol-4-yl]carbonyl}piperidin-3-yl)propan-2-ol;
2-((3R)-1-{[5-(2-fluorophenyl)isoxazol-4-yl]carbonyl}piperidin-3-yl)propan-2-ol;
2-((3R)-1-{[5-(4-fluorophenyl)isoxazol-4-yl]carbonyl}piperidin-3-yl)propan-2-ol;
2-((3S)-1-{[5-(4-fluorophenyl)isoxazol-4-yl]carbonyl}piperidin-3-yl)propan-2-ol;
2-((3R)-1-{[5-(1-naphthyl)isoxazol-4-yl]carbonyl}piperidin-3-yl)propan-2-ol;
2-((3S)-1-{[3-(2-methylphenyl)isoxazol-4-yl]carbonyl}piperidin-3-yl)propan-2-ol;
2-((3R)-1-{[3-(2-methylphenyl)isoxazol-4-yl]carbonyl}piperidin-3-yl)propan-2-ol;
2-((3R)-1-{[3-(2-chlorophenyl)isoxazol-4-yl]carbonyl}piperidin-3-yl)propan-2-ol;
2-((3S)-1-{[3-(2-chlorophenyl)isoxazol-4-yl]carbonyl}piperidin-3-yl)propan-2-ol;
2-((3R)-1-{[3-(2,4-dimethylphenyl)isoxazol-4-yl]carbonyl}piperidin-3-yl)propan-2-ol;
1-(1-{[3-(2-chlorophenyl)isoxazol-4-yl]carbonyl}piperidin-3-yl)ethanol; and
2-((3S)-1-{[3-(2,4-dimethylphenyl)isoxazol-4-yl]carbonyl}piperidin-3-yl)propan-2-ol.

Another aspect of the present invention is a compound of formula (I) for use in therapy. The compounds as defined above are potent and selective 11β-HSD1 inhibitors. As such, they are useful in the treatment or prevention of glaucoma. The invention thus includes the compounds of formula (I) for use in the treatment or prevention of glaucoma.

In another aspect, the invention includes the use of the compounds of formula (I) in the manufacture of a medicament for the treatment or prevention of glaucoma.

In yet another aspect, the invention includes a method for treatment or prevention of glaucoma, comprising administering to a human subject in need of such treatment an effective amount of a compound of formula (I).

In yet another aspect, the invention provides a pharmaceutical formulation comprising a compound of the formula (I) as active ingredient, in combination with a pharmaceutically acceptable diluent or carrier. The said pharmaceutical formulation is useful in the treatment or prevention glaucoma.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

### DEFINITIONS

The following definitions shall apply throughout the specification and the appended claims.

Unless otherwise stated or indicated, the term “C₁₋₈-alkyl” denotes a straight or branched alkyl group having from 1 to 8 carbon atoms. Examples of said C₁₋₈-alkyl include methyl, ethyl, n-propyl, iso-propyl, n-butyl, iso-butyl, sec-butyl, t-butyl, and straight- and branched-chain pentyl, hexyl, heptyl and octyl. For parts of the range “C₁₋₈-alkyl” all subgroups thereof are contemplated such as C₁₋₇-alkyl, C₁₋₆-alkyl, C₁₋₅-alkyl, C₁₋₄-alkyl, C₁₋₃-alkyl, C₁₋₂-alkyl, C₂₋₈-alkyl, C₂₋₇-alkyl, C₂₋₆-alkyl, C₂₋₅-alkyl, C₂₋₄-alkyl, C₂₋₃-alkyl, C₃₋₈-alkyl, C₃₋₇-alkyl, etc.

Unless otherwise stated or indicated, the term “hydroxy-C₁₋₈-alkyl” denotes a straight or branched C₁₋₈-alkyl group that has a hydrogen atom thereof replaced with OH. Examples of said hydroxy-C₁₋₈-alkyl include hydroxymethyl, 2-hydroxyethyl, 2-hydroxypropyl, 2-hydroxy-2-methylpropyl and 1-hydroxy-1-methylethyl.

Unless otherwise stated or indicated, the term “C₁₋₈-alkoxy” denotes a straight or branched C₁₋₈-alkyl group attached to the remainder of the molecule through oxygen. Examples of said C₁₋₈-alkoxy include methoxy, ethoxy, n-propoxy, iso-propoxy, n-butoxy, iso-butoxy, sec-butoxy, t-butoxy, and straight- and branched-chain pentoxy, hexoxy, heptoxy and octoxy. For parts of the range “C₁₋₈-alkoxy” all subgroups thereof are contemplated such as C₁₋₇-alkoxy, C₁₋₆-alkoxy, C₁₋₅-alkoxy, C₁₋₄-alkoxy, C₁₋₃-alkoxy, C₁₋₂-alkoxy, C₂₋₈-alkoxy, C₂₋₇-alkoxy, C₂₋₆-alkoxy, C₂₋₅-alkoxy, C₂₋₄-alkoxy, C₂₋₃-alkoxy, C₃₋₈-alkoxy, C₃₋₇-alkoxy, etc.

Unless otherwise stated or indicated, the term “C₁₋₈-alkoxy-C₁₋₈-alkyl” denotes a straight or branched C₁₋₈-alkyl group that has a hydrogen atom thereof replaced with a straight or branched C₁₋₈-alkoxy group. Examples of said C₁₋₈-alkoxy-C₁₋₈-alkyl include methoxymethyl, 1-methoxyethyl, 2-methoxyethyl and 2-ethoxyethyl.

Unless otherwise stated or indicated, the term “hydroxy-C₁₋₈-alkoxy” denotes a straight or branched C₁₋₈-alkoxy group that has a hydrogen atom thereof replaced with OH. Examples of said hydroxy-C₁₋₈-alkoxy include hydroxymethoxy, 2-hydroxyethoxy and 2-hydroxypropoxy.

Unless otherwise stated or indicated, the term “C₁₋₈-alkoxy-C₁₋₈-alkoxy” denotes a straight or branched C₁₋₈-alkoxy group that has a hydrogen atom thereof replaced with a straight or branched C₁₋₈-alkoxy group. Examples of said C₁₋₈-alkoxy-C₁₋₈-alkoxy include methoxymethoxy, 2-methoxyethoxy and 3-methoxypropoxy.

Unless otherwise stated or indicated, the term “C₃₋₈-cycloalkyl” denotes a monocyclic saturated hydrocarbon ring system having 3 to 8 carbon atoms. Examples of C₃₋₈-cycloalkyl include cyclopropyl, cyclobutyl, cyclopentyl, cyclohexyl, cycloheptyl, and cyclooctyl. For parts of the range “C₃₋₈-cycloalkyl” all subgroups thereof are contemplated such as C₃₋₇-cycloalkyl, C₃₋₆-cycloalkyl, C₃₋₅-cycloalkyl, C₃₋₄-cycloalkyl, C₄₋₈-cycloalkyl, C₄₋₇-cycloalkyl, C₄₋₆-cycloalkyl, C₄₋₅-cycloalkyl, C₅₋₈-cycloalkyl, C₅₋₇-cycloalkyl, C₆₋₈-cycloalkyl, and C₆₋₇-cycloalkyl.

Unless otherwise stated or indicated, the term “hydroxy-C₃₋₈-cycloalkyl” denotes a C₃₋₈-cycloalkyl group that has a hydrogen atom thereof replaced with OH. Examples of said hydroxy-C₃₋₈-cycloalkyl include 3-hydroxycyclopentyl and 4-hydroxycyclohexyl.

Unless otherwise stated or indicated, the term “C₁₋₈-alkoxy-C₃₋₈-cycloalkyl” denotes a C₃₋₈-cycloalkyl group that has a hydrogen atom thereof replaced with a straight or branched C₁₋₈-alkoxy group. Examples of said C₁₋₈-alkoxy-C₃₋₈-cycloalkyl include 3-methoxycyclopentyl and 4-methoxycyclohexyl.

Unless otherwise stated or indicated, the term “C₃₋₈-cycloalkyloxy” denotes a C₃₋₈-cycloalkyl group attached to the remainder of the molecule through oxygen. Examples of said C₃₋₈-cycloalkyloxy include cyclopropyloxy, cyclopentyloxy and cyclohexyloxy.

Unless otherwise stated or indicated, the term “hydroxy-C₃₋₈-cycloalkyloxy” denotes a C₃₋₈-cycloalkyloxy group that has a hydrogen atom thereof replaced with OH. Examples of said hydroxy-C₃₋₈-cycloalkyloxy include 3-hydroxycyclopentyloxy and 4-hydroxy-cyclohexyloxy.

Unless otherwise stated or indicated, the term “C₁₋₈-alkoxy-C₃₋₈-cycloalkyloxy” denotes a C₃₋₈-cycloalkyloxy group that has a hydrogen atom thereof replaced with a straight or branched C₁₋₈-alkoxy group. Examples of said C₁₋₈-alkoxy-C₃₋₈-cycloalkyloxy include 3-methoxycyclopentyloxy and 4-methoxycyclohexyloxy.

When two substituents R¹ described herein, together with the carbon atoms they are attached to, form a 5- or 6-membered aromatic or non-aromatic ring, said ring can optionally contain one or more heteroatoms selected from O and N. Examples of such bivalent substituents R¹ include —CH═CH—CH═CH—, —O—CH₂—O— (methylenedioxy) and —O—CH₂—CH₂—O— (ethylenedioxy).

“Halogen” refers to fluorine, chlorine, bromine or iodine.
“Hydroxy” refers to the —OH radical.
“Cyano” refers to the —CN radical.
“CF₃” refers to the trifluoromethyl radical.
“OCF₃” refers to the trifluoromethoxy radical.

“Optional” or “optionally” means that the subsequently described event or circumstance may but need not occur, and that the description includes instances where the event or circumstance occurs and instances in which it does not.

“Pharmaceutically acceptable” means being useful in preparing a pharmaceutical composition that is generally safe, non-toxic and neither biologically nor otherwise undesirable and being useful for human pharmaceutical use.

“Treatment” as used herein includes prophylaxis of the named disorder or condition, or amelioration or elimination of the disorder once it has been established.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect).

“Prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis. The prodrug compound usually offers advantages of solubility, tissue compatibility or delayed release in a mammalian organism (see Silverman, R. B., The Organic Chemistry of Drug Design and Drug Action, 2^{nd} Ed., Elsevier Academic Press (2004), pp. 498-549). Prodrugs of a compound of the invention may be prepared by modifying functional groups, such as a hydroxy group, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups.

Throughout the specification and the appended claims, a given chemical formula or name shall also encompass all salts, hydrates, solvates, N-oxides and prodrug forms thereof. Further, a given chemical formula or name shall encompass all tautomeric and stereoisomeric forms thereof. Stereoisomers include enantiomers and diastereomers. Enantiomers can be present in their pure forms, or as racemic (equal) or unequal mixtures of two enantiomers. Diastereomers can be present in their pure forms, or as mixtures of diastereomers. Diastereomers also include geometrical isomers, which can be present in their pure cis or trans forms or as mixtures of those.

The compounds of formula (I) may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

### Compositions

For clinical use, the compounds of the invention are formulated into pharmaceutical formulations for ocular administration. It will be appreciated that compounds of the invention may be administered together with a physiologically acceptable carrier, excipient, or diluent.

The preparation of a pharmacological composition that contains active ingredients dissolved, dispersed or suspended therein is well understood in the art. Typically such compositions are prepared as sterile compositions for instillation (oculoguttae) either as liquid solutions or suspensions, aqueous or non-aqueous, however, solid forms suitable for solution, dispersion or suspensions, in liquid prior to use can also be prepared. The preparation can also be emulsified.

The active ingredient may be mixed with excipients which are pharmaceutically acceptable and compatible with the active ingredient and in amounts suitable for use in the therapeutic methods described herein. If desired, the composition may contain auxiliary substances such as wetting or emulsifying agents, pH buffering agents and the like which enhance the effectiveness of the active ingredient.

Pharmaceutically acceptable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate buffered saline. Still further aqueous carriers can contain more than one buffer salt, as well as salts such as sodium and potassium chlorides, dextrose, propylene glycol, polyethylene glycol and other solutes. Liquid compositions can also contain liquid phases in addition to and to the exclusion of water. Examples of such additional liquid phases are glycerin, vegetable oils, organic esters and water-oil emulsions.

Preferably, the pharmaceutical composition comprises one or more agents useful as solubiliser, emulsifier and/or penetration enhancer. Such agents, which are well known in the art, include e.g. agents sold under the name Cremophor® (BASF). An example is Cremophor® RH 40 (polyoxol castor oil; CAS No. 61788-85-0).

### Preparation of Compounds of the Invention

The compounds of formula (I) above may be prepared by, or in analogy with, conventional methods. The preparation of intermediates and compounds according to the examples of the present invention may in particular be illuminated by the following Schemes 1 and 2. Definitions of variables in the structures in the schemes herein are commensurate with those of corresponding positions in the formulae delineated herein.

Starting from the appropriate acetophenone (II), the 5-(phenyl)isoxazole-4-carboxylic acid methyl ester (V) can easily be obtained in a few synthetic steps. After hydrolysis of the ester, the carboxylic acid (VI) is activated by treatment with TBTU, or transformed into the corresponding acid chloride, and allowed to react with the appropriate cyclic amine (VII), resulting in the formation of the desired compound of formula (I). This is generally represented in Scheme 1.

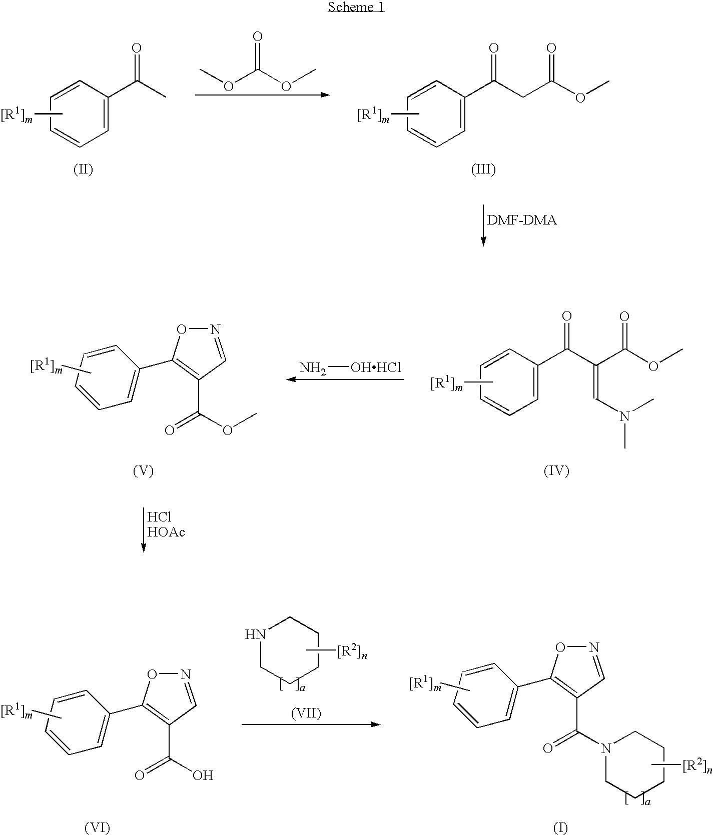

wherein R¹-R², a, n and m are as defined in formula (I)

Scheme 2 shows the preparation of the isomeric isoxazole compounds of formula (I). Starting from an appropriately substituted benzaldehyde (VIII), the 3-(phenyl)isoxazole-4-carboxylic acid ethyl ester (XI) is obtained in three steps. The compounds of formula (I) are then easily formed by hydrolysis of ester (XI) and condensation of the resulting acid (XII) with the appropriate cyclic amine (VII), in the presence of TBTU as the activating agent, or via transformation of the acid to the corresponding acid chloride.

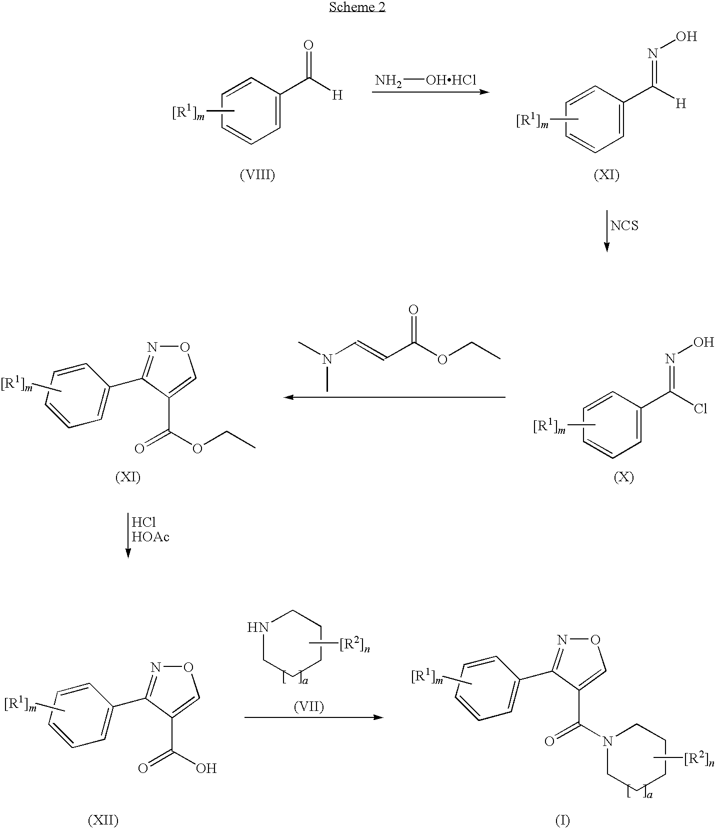

wherein R¹-R², a, n and m are as defined in formula (I)

The necessary starting materials for preparing the compounds of formula (I) are either commercially available, or may be prepared by methods known in the art.

The processes described below in the experimental section may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. Examples of protecting groups are t-butoxycarbonyl(Boc), benzyl and trityl (triphenylmethyl). The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The following abbreviations have been used:
DMF-DMA dimethylformamide dimethylacetal
EtOAc ethyl acetate
ESI Electrospray Ionization
h Hours
HPLC High Performance Liquid Chromatography
HRMS High Resolution Mass Spectrometry
LCMS Liquid Chromatography-Mass Spectrometry
M Molar
MeCN Acetonitrile
MeOH Methanol
min Minutes
MS Mass Spectrometry
NCS N-chlorosuccinimide
NEt₃ Triethylamine
TBTU 2-(1H-Benzotriazole-1-yl)-1,1,3,3-tetramethyluronium tetrafluoroborate
THF Tetrahydrofuran

The recitation of a listing of chemical groups in any definition of a variable herein includes definitions of that variable as any single group or combination of listed groups. The recitation of an embodiment herein includes that embodiment as any single embodiment or in combination with any other embodiments or portions thereof.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All references and publications cited herein are hereby incorporated by reference in their entirety.

### EXAMPLES AND INTERMEDIATE COMPOUNDS

### Experimental Methods

All reagents were commercial grade and were used as received without further purification, unless otherwise specified. Reagent grade solvents were used in all other cases, unless otherwise specified. Preparative HPLC/MS was performed on a Waters/Micromass Platform ZQ system and preparative HPLC/UV was performed on a Gilson system in accordance to the experimental details specified in the examples. Analytical HPLC/MS was performed using an Agilent 1100/1200 Series Liquid Chromatograph/Mass Selective Detector (MSD) (Single Quadrupole) (1946A/1946C/1956C/6110) equipped with an electrospray interface. Preparative flash chromatography was performed on Merck silica gel 60 (230-400 mesh). Microwave reactions were performed with a Personal Chemistry Smith Creator or Personal Chemistry Smith Optimizer using 0.5-2 mL or 2-5 mL Smith Process Vials fitted with aluminum caps and septa. High-resolution mass spectra (HRMS) were obtained on an Agilent MSD-TOF connected to an Agilent 1100 HPLC system. During the analyses the calibration was checked by two masses and automatically corrected when needed. Spectra were acquired in positive electrospray mode. The acquired mass range is m/z 100-1100. Profile detection of the mass peaks was used. The compounds were named using ACD Name 6.0.

### Intermediate 1

### 5-(2-methylphenyl)isoxazole-4-carboxylic acid

### Step 1: Methyl 3-(2-methylphenyl)-3-oxopropanoate

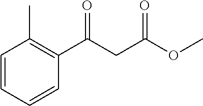

2-Methyl acetophenone (20 g, 149 mmol) was dissolved in toluene (20 mL) and added slowly to a stirred slurry of NaH (7.16 g, 298 mmol) and dimethyl carbonate (14.8 g, 164 mmol) in toluene (ca 150 mL) at room temperature. The reaction was monitored by LCMS. When the starting acetophenone was consumed, MeOH (ca 10 mL) was added over ca 10 min, while stirring, followed by ca 50 mL of crushed ice. The mixture was diluted with CH₂Cl₂ and acidified by aqueous HCl (9-12 M). The organic phase was separated and the aqueous phase extracted with CH₂Cl₂. The combined organic phases were dried (Na₂SO₄) and the solvent evaporated to furnish 27.2 g of the title compound (>95% pure). MS m/z 193 [m+1].

### Step 2: Methyl (2Z)-3-(dimethylamino)-2-(2-methylbenzoyl)acrylate

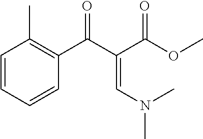

Methyl 3-(2-methylphenyl)-3-oxopropanoate (27.1 g, 141 mmol) was dissolved in toluene (100 mL). DMF-DMA (17.6 g, 148.0 mmol) was added and the reaction mixture was stirred at 70° C. When the starting material was consumed (monitored by LCMS), the reaction mixture was allowed to cool to room temperature, and the solvent was evaporated to furnish 34.1 g of the title compound (>95% pure) which was used in the next step. MS m/z 248 [m+1].

### Step 3: Methyl 5-(2-methylphenyl)isoxazole-4-carboxylate

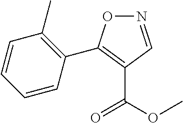

Methyl (2Z)-3-(dimethylamino)-2-(2-methylbenzoyl)acrylate (34.0 g, 138 mmol) and hydroxylamine hydrochloride (10.1 g, 145 mmol) were dissolved in MeOH (150 mL) and the resulting solution was stirred at room temperature. The reaction was monitored by HPLC. After 18 h, 90% of the solvent was evaporated and the residue was dissolved in CH₂Cl₂, washed with water and dried (Na₂SO₄). The solvent was evaporated to furnish 28.5 g (>95% pure) of the title compound. MS m/z 218 [m+1].

### Step 4: 5-(2-methylphenyl)isoxazole-4-carboxylic acid

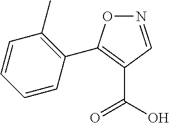

Methyl 5-(2-methylphenyl)isoxazole-4-carboxylate (8.00 g, 36.8 mmol) was dissolved in HOAc (40 mL). HCl (conc) was added while stirring at room temperature until turbidity was observed (ca 40 mL). The reaction mixture was stirred at 70° C. and monitored by HPLC. After 18 h the reaction was allowed to cool to room temperature, diluted with water (100 mL) and extracted with CH₂Cl₂ (2×50 mL). The combined organic phases were washed with water (50 mL). The organic phase was neutralized with K₂CO₃ and extracted with aq K₂CO₃ solution (3×100 mL). The combined alkaline aqueous phases were washed with CH₂Cl₂ (ca 50 mL), acidified with aq HCl (conc), and extracted with CH₂Cl₂ (3×100 mL). The combined organic phases were dried (Na₂SO₄) and the solvent evaporated to furnish 6.51 g of a beige solid (>98% pure). MS m/z 204 [m+1].

### Intermediate 2-12

Starting from the appropriately substituted acetophenone, Intermediates 2-12 were prepared following the procedure as outlined for Intermediate 1.

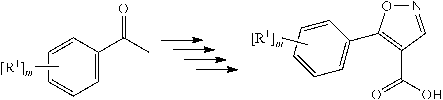

| Intermediate | [R¹]ₘ | MS m/z [m + 1] |
|---|---|---|
| 2 | 3-Me | 204 |
| 3 | 4-Me | 204 |
| 4 | 2-Cl | 224 |
| 5 | 3-Cl | 224 |
| 6 | 4-Cl | 224 |
| 7 | 2-Me, 3-Cl | 238 |
| 8 | 3,4-di-Me | 218 |
| 9 | 2,4-di-Me | 218 |
| 10 | 2-F | 208 |
| 11 | 4-F | 208 |
| 12 | 2,3-CH═CH—CH═CH— | 240 |

### Intermediate 13

### 3-(2,4-dimethylphenyl)isoxazole-4-carboxylic acid

### Step 1: 2,4-dimethylbenzaldehyde oxime

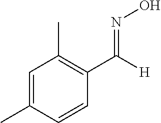

A solution of 2,4-dimethylbenzaldehyde (10.0 g; 74.5 mmol), hydroxylamine hydrochloride (7.73 g; 111 mmol) and pyridine (10 mL) in MeOH (75 mL) was stirred at room temperature overnight. The solvent was evaporated and the crude mixture was dissolved in CH₂Cl₂ (150 mL), washed with water (4×25 mL) and dried (Na₂SO₄). The solvent was evaporated to furnish 10.1 g of a transparent oil which solidified at room temperature and was used in the next step. MS m/z 150 [m+1].

### Step 2: 2,4-dimethyl-N-hydroxybenzenecarboximidoyl chloride

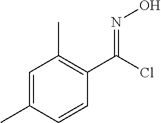

To a stirred solution of 2,4-dimethylbenzaldehyde oxime (6.12 g, 41.1 mmol) in DMF (100 mL) was added a solution of N-chlorosuccinimide (6.30 g, 47.2 mmol) in DMF in small portions over 90 min at room temperature. The reaction mixture was stirred overnight and was then diluted with diethyl ether and washed with ice water and dried (MgSO₄). The solvent was evaporated to give 6.92 g of the title compound as a yellow oil. MS m/z 184 [m+1].

### Step 3: Ethyl 3-(2,4-dimethylphenyl)isoxazole-4-carboxylate

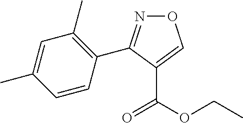

To an ice-cold solution of ethyl-3-(dimethylamino)acrylate (1.56 g, 10.9 mmol) in diethyl ether (30 mL) was added trimethylamine (1.52 ml, 10.9 mmol) followed by drop wise addition (over 1 h) of 2,4-dimethyl-N-hydroxybenzenecarboximidoyl chloride (2.012 g, 10.96 mmol) dissolved in diethyl ether (15 mL). A white precipitate formed and additional diethyl ether (50 mL) was added. The ice bath was removed and the reaction mixture was allowed to stir at 22° C. overnight. The solids were removed by filtration and the organic phase was washed with 5% aq HOAc solution (3×), dried (MgSO₄) and the solvent was evaporated to furnish 2.54 g of the title compound. MS m/z 246 [m+1].

### Step 4: 3-(2,4-dimethylphenyl)isoxazole-4-carboxylic acid

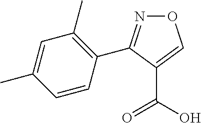

A solution of ethyl 3-(2,4-dimethylphenyl)isoxazole-4-carboxylate (890 mg, 3.63 mmol) in HOAc (10 mL) and 12M aq HCl (5 mL) was heated at 110° C. for 70 min in a microwave reactor. The reaction mixture was concentrated to circa half the volume and diluted with toluene. The aqueous phase was removed and the organic phase extracted with 1M aq HCl solution. The combined aqueous phases were extracted with toluene and the organic phases combined and concentrated. The residue was purified by flash chromatography (CH₂Cl₂/MeOH 9:1) to give 723 mg of the title compound as a brown oil. MS m/z 224 [m+1].

### Intermediate 14-16

Starting from the appropriately substituted benzaldehyde, Intermediates 14-16 were prepared following the procedure as outlined for Intermediate 13.

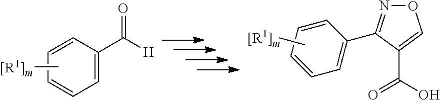

| Intermediate | [R¹]ₘ | MS m/z [m + 1] |
|---|---|---|
| 14 | 2-Me | 204 |
| 15 | 2-Cl | 224 |
| 16 | 4-Me | 204 |

### Intermediate 17

### 2-[(3S)-piperidin-3-yl]propan-2-ol hydrochloride

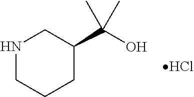

A solution of ethyl(S)-piperidine-3-carboxylate (25.0 g, 159 mmol) in CH₂Cl₂ (25 mL) was added dropwise to a stirred solution of di-tert-butyl dicarbonate (36.5 g, 167 mmol) in CH₂Cl₂ (150 mL) at room temperature. The reaction was allowed to stir overnight. The crude mixture was washed with sat aq NaHCO₃ solution and then stirred for 90 min with 10% aq NH₃ solution (ca 30 mL). The organic phase was separated, dried (Na₂SO₄) and the solvent was evaporated to furnish 39.7 g of 1-tert-butyl 3-ethyl (3S)-piperidine-1,3-dicarboxylate as a transparent oil, which solidified on standing.

A solution of 1-tert-butyl 3-ethyl (3S)-piperidine-1,3-dicarboxylate (16.0 g, 62.2 mmol) in dry THF (100 mL) was added dropwise over 1 h to a stirred solution of MeMgBr in THF/toluene (100 mL, 1.4 M, 140 mmol). The reaction temperature was kept below 25° C. by cooling with ice. The reaction mixture was allowed to stir overnight and was quenched is by dropwise addition of water (ca 5 mL) followed by aq HCl (6 M) until acidic indication by pH-paper. The organic phase was separated and the solvent evaporated. The residue was dissolved in CH₂Cl₂, washed with water (ca 50 mL) and dried (Na₂SO₄) and the solvent was evaporated to furnish 14.7 g of a pale yellow oil. The crude product was dissolved in EtOAc/HCl(g) (100 mL; prepared from bubbling HCl(g) into EtOAc) and stirred at room temperature over night. The solvent was evaporated and the crude was dissolved in CH₂Cl₂ (100 mL) and stirred for 5 min. The solvent was evaporated (repeated once) and the crude was warmed to 50° C. in vacuo for 3 h. The crude was triturated with diethyl ether resulting in the formation of white crystals which were filtered and dried to furnish 6.41 g of the title compound. MS m/z 144 [m+1].

### Intermediate 18

### 2-[(3R)-piperidin-3-yl]propan-2-ol hydrochloride

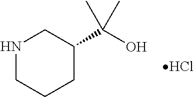

The title compound was prepared from ethyl(R)-piperidine-3-carboxylate (25.0 g, 159 mmol) and di-tert-butyl dicarbonate (36.5 g, 167 mmol) as described in the procedure for intermediate 17, to furnish 6.88 g of the product as a white solid. MS m/z 144 [m+1].

### Intermediate 19

### 2-[piperidin-3-yl]propan-2-ol hydrochloride

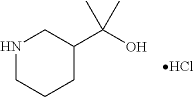

The title compound was prepared from ethyl piperidine-3-carboxylate (6.837 g, 47.75 mmol) and di-tert-butyl dicarbonate (10.42 g, 47.75 mmol) as described in the procedure for intermediate 17, to furnish 4.85 g of the product as a white solid. MS m/z 144 [m+1].

### Intermediate 20

### 1-(piperidin-3-yl)etan-1-ol

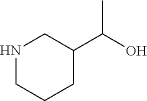

A mixture of 1-(pyridin-3-yl)ethanol (249 mg, 2.03 mmol), ammonium formate (557 mg, 8.83 mmol) and Pd/C (10%, 65.5 mg) in EtOH (15 mL) was heated to 110° C. for 20 min in a microwave reactor. An additional amount of ammonium formate (412 mg, 6.53 mmol) was added and the reaction mixture was heated to 110° C. for 20 min. The reaction mixture was filtered and the solvent evaporated to furnish 165 mg the title compound. MS m/z 130 [m+1].

### Example 1

### 2-(1-{[5-(2-methylphenyl)isoxazol-4-yl]carbonyl}piperidin-3-yl)propan-2-ol

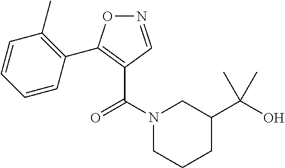

A solution of 5-(2-methylphenyl)isoxazole-4-carboxylic acid (Intermediate 1; 103 mg, 0.51 mmol) and TBTU (195 mg, 0.607 mmol) in CH₂Cl₂ (5 mL) was added to a mixture of 2-piperidin-3-ylpropan-2-ol hydrochloride (Intermediate 19; 116 mg, 0.645 mmol) and triethylamine (0.142 mL, 1.01 mmol) in CH₂Cl₂ (2 mL). The mixture was stirred at room temperature for 30 min. The mixture was diluted with diethyl ether, washed with sat aq NaHCO₃ solution, 5% aq HOAc solution, and then dried (MgSO₄). The solvent was evaporated and the residue was purified by flash chromatography (diethyl ether/petroleum ether/MeOH 70:25:5) to give 145 mg of the title compound.

HRMS (ESI+) calcd for C₁₉H₂₄N₂O₃ 328.1786, found 328.1784.

### Example 2

### 2-((3R)-1-{[5-(2-methylphenyl)isoxazol-4-yl]carbonyl}piperidin-3-yl)propan-2-ol

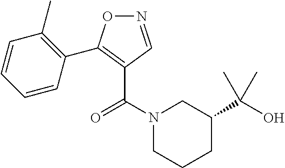

A solution of 5-(2-methylphenyl)isoxazole-4-carboxylic acid (Intermediate 1; 102 mg, 0.507 mmol) and TBTU (196 mg, 0.611 mmol) in CH₂Cl₂ (5 mL) was added to a mixture of 2-[(3R)-piperidin-3-yl]propan-2-ol hydrochloride (Intermediate 18; 84.1 mg, 0.471 mmol) and triethylamine (0.142 mL, 1.01 mmol) in CH₂Cl₂ (2 mL). The mixture was stirred overnight at room temperature, and then diluted with EtOAc, washed with aq NaHCO₃ solution, 5% aq HOAc solution and dried (MgSO₄). The solvent was evaporated and the residue was purified by flash chromatography (diethyl ether/petroleum ether/MeOH 60:35:5) to give 101 mg of the title compound.

HRMS (ESI+) calcd for C₁₉H₂₄N₂O₃ 328.1786, found 328.1790.

### Example 3

### 2-((3S)-1-{[5-(2-methylphenyl)isoxazol-4-yl]carbonyl}piperidin-3-yl)propan-2-ol

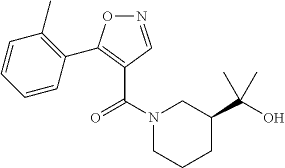

A solution of 5-(2-methylphenyl)isoxazole-4-carboxylic acid (Intermediate 1; 64.0 mg, 0.36 mmol) and TBTU (101 mg, 0.315 mmol) in CH₂Cl₂ (2 mL) was added to a mixture of 2-[(3S)-piperidin-3-yl]propan-2-ol hydrochloride (Intermediate 17; 54.1 mg, 0.379 mmol) and triethylamine (0.048 mL, 0.341 mmol) in CH₂Cl₂ (2 mL). The mixture was stirred at room temperature for 1 hr. The crude product was purified by preparative HPLC (Xterra C18, 10 mM NH₄HCO₃ (pH 10)-CH₃CN) (5-30% MeCN) to give 23 mg of the title compound.

HRMS (ESI+) calcd for C₁₉H₂₄N₂O₃ 328.1786, found 328.1788.

### Example 4

### 2-(1-{[5-(3-methylphenyl)isoxazol-4-yl]carbonyl}piperidin-3-yl)propan-2-ol

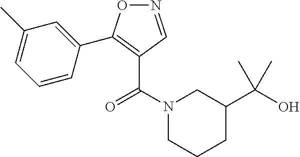

A solution of 5-(3-methylphenyl)isoxazole-4-carboxylic acid (Intermediate 2; 112 mg, 0.551 mmol) and TBTU (194 mg, 0.607 mmol) in CH₂Cl₂ (5 mL) was added to a mixture of 2-piperidin-3-ylpropan-2-ol hydrochloride (Intermediate 19; 136 mg, 0.757 mmol) and triethylamine (0.196 mL, 1.41 mmol) in CH₂Cl₂ (2 mL). The mixture was stirred at room temperature for 30 min, diluted with diethyl ether, washed with sat aq NaHCO₃ solution, 5% aq HOAc solution and then dried (MgSO₄). The solvent was evaporated and the residue was purified by flash chromatography (diethyl ether/petroleum ether/MeOH 70:25:5) to give 174 mg of the title compound.

HRMS (ESI+) calcd for C₁₉H₂₄N₂O₃ 328.1786, found 328.1785.

### Example 5

### 2-((3S)-1-{[5-(4-methylphenyl)isoxazol-4-yl]carbonyl}piperidin-3-yl)propan-2-ol

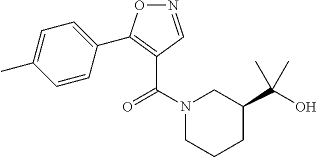

DMF (3 drops) was added to a solution of 5-(4-methylphenyl)isoxazole-4-carboxylic acid (Intermediate 3; 1.10 g, 5.41 mmol) in SOCl₂ (8 mL). The mixture was warmed to 110° C. for 30 min and then concentrated in vacuo. A cold (−20° C.) solution of the acid chloride in CH₂Cl₂ (20 mL) was added to a solution of 2-[(3S)-piperidin-3-yl]propan-2-ol hydrochloride (Intermediate 17; 0.973 g, 5.41 mmol) and triethylamine (1.49 mL, 11.8 mmol) in CH₂Cl₂ (10 mL) at −20° C. The reaction mixture was stirred for 30 min and then diluted with CH₂Cl₂. The mixture was washed with 0.5 M aq HCl solution (3×15 mL), dried (Na₂SO₄) and the solvent was evaporated to furnish a clear syrup/oil. The syrup was dissolved in diethyl ether and the solvent was evaporated at room temperature. The solid part was triturated in diethyl ether resulting in formation of 750 mg of the title compound as a white solid.

HRMS (ESI+) calcd for C₁₉H₂₄N₂O₃ 328.1786, found 328.1797.

### Example 6

### 2-((3R)-1-{[5-(2-chlorophenyl)isoxazol-4-yl]carbonyl}piperidin-3-yl)propan-2-ol

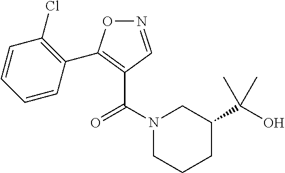

A solution of 5-(2-chlorophenyl)isoxazole-4-carboxylic acid (Intermediate 4; 148 mg, 0.664 mmol) and TBTU (264 mg, 0.822 mmol) in CH₂Cl₂ (10 mL) was added to a mixture of 2-[(3R)-piperidin-3-yl]propan-2-ol hydrochloride (Intermediate 18; 169 mg, 0.940 mmol) and NEt₃ (236 μL, 172 mg, 1.70 mmol) in CH₂Cl₂ (5 mL). The mixture was stirred at room temperature for 30 min. The solvent was evaporated and the residue was purified by flash chromatography (petroleum ether/EtOAc (1:1, 1-6% MeOH)) to afford 179 mg of the title compound.

HRMS (ESI+) calcd for C₁₈H₂₁ClN₂O₃ 348.1241, found 348.1247.

### Example 7

### 2-((3S)-1-{[5-(2-chlorophenyl)isoxazol-4-yl]carbonyl}piperidin-3-yl)propan-2-ol

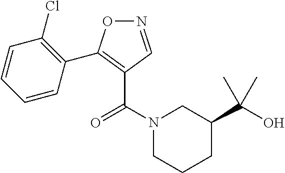

A mixture of 5-(2-chlorophenyl)isoxazole-4-carboxylic acid (Intermediate 4; 149 mg, 0.666 mmol) and TBTU (246 mg, 0.766 mmol) in CH₂Cl₂ (5 mL) was added to a solution of 2-[(3S)-piperidin-3-yl]propan-2-ol hydrochloride (Intermediate 17; 147 mg, 0.818 mmol) and NEt₃ (190 μL, 1.34 mmol) in CH₂Cl₂ (2 mL). The mixture was stirred at room temperature for 30 min and diluted with EtOAc, washed with sat aq NaHCO₃ solution, brine, and dried (MgSO₄). The solvent was evaporated and the residue was purified by flash chromatography (petroleum ether/EtOAc 1:1, gradient 1-6% MeOH) to give 23.1 mg of the title compound.

HRMS (ESI+) calcd for C₁₈H₂₁ClN₂O₃ 348.1241, found 348.1249.

### Example 8

### 2-((3S)-1-{[5-(3-chlorophenyl)isoxazol-4-yl]carbonyl}piperidin-3-yl)propan-2-ol

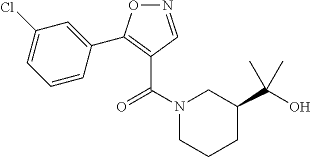

A solution of 5-(3-chlorophenyl)isoxazole-4-carboxylic acid (Intermediate 5; 67 mg, 0.30 mmol) and TBTU (100 mg, 0.312 mmol) in CH₂Cl₂ (5 mL) was added to a mixture of 2-[(3S)-piperidin-3-yl]propan-2-ol hydrochloride (Intermediate 17; 57 mg, 0.32 mmol) and NEt₃ (120 μL, 1.34 mmol) in CH₂Cl₂ (2 mL). The reaction mixture was stirred at room temperature for 30 min. The solvent was evaporated and the residue purified by preparative HPLC (Xterra C18, 10 mM NH₄HCO₃ (pH 10)-CH₃CN) (5-30% MeCN) to give 12 mg of the title compound.

HRMS (ESI+) calcd for C₁₈H₂₁ClN₂O₃ 348.1241, found 348.1244.

### Example 9

### 2-((3R)-1-{[5-(4-chlorophenyl)isoxazol-4-yl]carbonyl}piperidin-3-yl)propan-2-ol

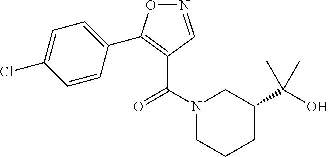

A solution of 5-(4-chlorophenyl)isoxazole-4-carboxylic acid (Intermediate 6; 117 mg, 0.524 mmol) and TBTU (195.7 mg, 0.609 mmol) in CH₂Cl₂ (5 mL) was added to a mixture of 2-[(3R)-piperidin-3-yl]propan-2-ol hydrochloride (Intermediate 18; 107 mg, 0.595 mmol) and triethylamine (150 μL, 1.05 mmol) in CH₂Cl₂ (2 mL). The mixture was stirred at room temperature for 30 min. The mixture was diluted with diethyl ether, washed with sat aq NaHCO₃ solution, 5% aq HOAc solution and dried (MgSO₄). The solvent was evaporated and the residue was purified by flash chromatography (diethyl ether/petroleum ether/MeOH 70:25:5) to give 120 mg of the title compound.

HRMS (ESI+) calcd for C₁₈H₂₁ClN₂O₃ 348.1241, found 348.1247.

### Example 10

### 2-((3S)-1-{[5-(4-chlorophenyl)isoxazol-4-yl]carbonyl}piperidin-3-yl)propan-2-ol

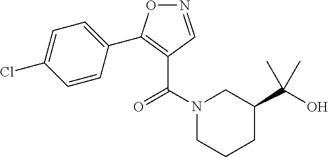

A solution of 5-(3-chlorophenyl)isoxazole-4-carboxylic acid (Intermediate 6; 57 mg, 0.30 mmol) and TBTU (80 mg, 0.249 mmol) in CH₂Cl₂ (5 mL) was added to a mixture of 2-[(3S)-piperidin-3-yl]propan-2-ol hydrochloride (Intermediate 17; 147 mg, 0.818 mmol) and NEt₃ (120 μL, 1.34 mmol) in CH₂Cl₂ (2 mL). The reaction mixture was stirred at room temperature for 30 min. The solvent was evaporated and the residue purified by preparative HPLC (Xterra C18, 10 mM NH₄HCO₃ (pH 10)-CH₃CN) (5-30% MeCN) to give 19 mg of the title compound.

HRMS (ESI+) calcd for C₁₈H₂₁ClN₂O₃ 348.1241, found 348.1245.

### Example 11

### 2-(1-{[5-(2-chlorophenyl)isoxazol-4-yl]carbonyl}piperidin-2-yl)ethanol

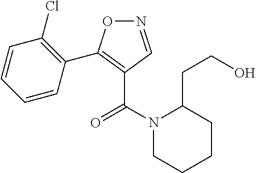

2-(2-hydroxyethyl)piperidine (203 mg, 1.57 mmol) was added to a solution of 5-(2-chlorophenyl)isoxazole-4-carboxylic acid (Intermediate 4; 120 mg, 0.54 mmol) and TBTU (206 mg, 0.642 mmol) in CH₂Cl₂ (6 mL). The reaction was stirred at room temperature for 30 min. The mixture was diluted with diethyl ether, washed with sat aq NaHCO₃ solution, 5% aq HOAc solution and then dried (MgSO₄). The solvent was evaporated and the residue was purified by flash chromatography (diethyl ether/petroleum ether/MeOH 70:25:5) to give 5 mg of the title compound.

HRMS (ESI+) calcd for C₁₇H₁₉ClN₂O₃ 334.1084, found 334.1081.

### Example 12

### 2-(1-{[5-(3-chloro-2-methylphenyl)isoxazol-4-yl]carbonyl}piperidin-3-yl)propan-2-ol

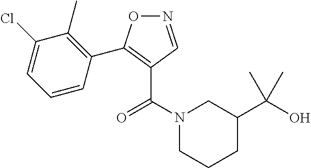

A solution of 5-(3-chloro-2-methylphenyl)isoxazole-4-carboxylic acid (Intermediate 7; 95.5 mg, 0.402 mmol) and TBTU (147 mg, 0.458 mmol) in CH₂Cl₂ (6 mL) was added to a mixture of 2-piperidin-3-ylpropan-2-ol hydrochloride (Intermediate 19; 84.0 mg, 0.467 mmol) and triethylamine (0.112 mL, 0.80 mmol) in CH₂Cl₂ (2 mL). The mixture was stirred at room temperature for 30 min. The mixture was diluted with diethyl ether, washed with sat aq NaHCO₃ solution, 5% aq HOAc solution and then dried (MgSO₄). The solvent was evaporated and the residue was purified by flash chromatography (diethyl ether/petroleum ether/MeOH 70:25:5) to give 63 mg of the title compound.

HRMS (ESI+) calcd for C₁₉H₂₃ClN₂O₃ 362.1397, found 362.1392.

### Example 13

### 2-(1-{[5-(3,4-dimethylphenyl)isoxazol-4-yl]carbonyl}piperidin-3-yl)propan-2-ol

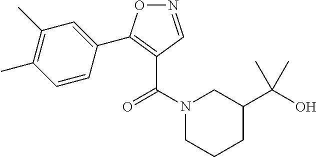

A solution of 5-(3,4-dimethylphenyl)isoxazole-4-carboxylic acid (Intermediate 8; 56.0 mg, 0.26 mmol) and TBTU (103 mg, 0.321 mmol) in CH₂Cl₂ (6 mL) was added to a mixture of 2-piperidin-3-ylpropan-2-ol (Intermediate 19; 52.2 mg, 0.290 mmol) and triethylamine (0.072 mL, 0.52 mmol) in CH₂Cl₂ (2 mL). The mixture was stirred at room temperature for 1 h. The mixture was diluted with diethyl ether, washed with sat aq NaHCO₃ solution, 5% aq HOAc solution, dried (MgSO₄) and then concentrated. The residue was purified by flash chromatography (diethyl ether/petroleum ether/MeOH 70:25:5) to give 46 mg of the title compound. HRMS (ESI+) calcd for C₂₀H₂₆N₂O₃ 342.1943, found 342.1941.

### Example 14

### 2-((3R)-1-{[5-(3,4-dimethylphenyl)isoxazol-4-yl]carbonyl}piperidin-3-yl)propan-2-ol

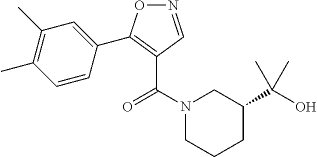

A solution of 5-(3,4-dimethylphenyl)isoxazole-4-carboxylic acid (Intermediate 8; 139 mg, 0.642 mmol) and TBTU (245 mg, 0.766 mmol) in CH₂Cl₂ (6 mL) was added to a mixture of 2-[(3R)-piperidin-3-yl]propan-2-ol (Intermediate 18; 140 mg, 0.779 mmol) and triethylamine (0.225 mL, 1.6 mmol) in CH₂Cl₂ (2 mL). The mixture was stirred at room temperature for 1 h, diluted with diethyl ether, washed with sat aq NaHCO₃ solution, 5% aq HOAc solution, dried (MgSO₄) and concentrated The residue was purified by flash chromatography (diethyl ether/petroleum ether/MeOH 70:25:5) to give 202 mg of the title compound.

HRMS (ESI+) calcd for C₂₀H₂₆N₂O₃ 342.1943, found 342.1946.

### Example 15

### 2-((3S)-1-{[5-(3,4-dimethylphenyl)isoxazol-4-yl]carbonyl}piperidin-3-yl)propan-2-ol

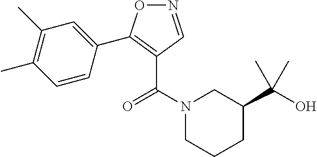

A solution of 5-(3,4-dimethylphenyl)isoxazole-4-carboxylic acid (Intermediate 8; 68.0 mg, 0.31 mmol) and TBTU (101 mg, 0.315 mmol) in CH₂Cl₂ (2 mL) was added to a mixture of 2-[(3S)-piperidin-3-yl]propan-2-ol hydrochloride (Intermediate 17; 54 mg, 0.30 mmol) and triethylamine (120 μL, 1.34 mmol) in CH₂Cl₂ (2 mL). The mixture was stirred at room temperature for 1 hr. The crude product was purified by preparative HPLC (Xterra C18, 10 mM NH₄HCO₃ (pH 10)-CH₃CN) (5-30% MeCN) to give 9 mg of the title compound.

HRMS (ESI+) calcd for C₂₀H₂₆N₂O₃ 342.1943, found 342.1949.

### Example 16

### 2-((3R)-1-{[5-(2,4-dimethylphenyl)isoxazol-4-yl]carbonyl}piperidin-3-yl)propan-2-ol

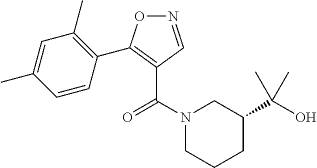

A solution of 5-(2,4-dimethylphenyl)isoxazole-4-carboxylic acid (Intermediate 9; 104 mg, 0.478 mmol) and TBTU (166 mg, 0.517 mmol) in CH₂Cl₂ (6 mL) was added to a mixture of 2-[(3R)-piperidin-3-yl]propan-2-ol (Intermediate 18; 92.5 mg, 0.515 mmol) and triethylamine (133 μL, 97 mg, 0.96 mmol) in CH₂Cl₂ (2 mL). The mixture was stirred at room temperature for 1 h. The solvent was evaporated and the residue was purified by flash chromatography (diethyl ether/petroleum ether/MeOH 70:26:4) to give 105 mg of the title compound.

HRMS (ESI+) calcd for C₂₀H₂₆N₂O₃ 342.1943, found 342.1945.

### Example 17

### 2-((3S)-1-{[5-(2,4-dimethylphenyl)isoxazol-4-yl]carbonyl}piperidin-3-yl)propan-2-ol

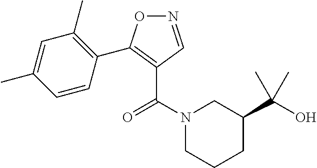

DMF (3 drops) was added to a solution of 5-(2,4-dimethylphenyl)isoxazole-4-carboxylic acid (Intermediate 9; 1.00 g, 4.60 mmol) in SOCl₂ (8 mL) and the mixture was warmed to 110° C. for 30 min and then concentrated in vacuo. A cold (−20° C.) solution of the acid chloride in CH₂Cl₂ (20 mL) was added to a solution of 2-[(3S)-piperidin-3-yl]propan-2-ol hydrochloride (Intermediate 17; 0.827 g, 4.60 mmol) and triethylamine (1.3 mL, 9.2 mmol) in CH₂Cl₂ (10 mL) at −70° C. The reaction mixture was stirred for 30 min at room temperature, diluted with CH₂Cl₂ and washed with 0.5 M aq HCl (3×15 mL) and then dried (Na₂SO₄). The solvent was evaporated to furnish a clear syrup/oil. The syrup was dissolved in diethyl ether and the solvent was evaporated at room temperature. The solid part was triturated with diethyl ether resulting in formation of 1.32 g of the title compound as a beige solid.

HRMS (ESI+) calcd for C₂₀H₂₆N₂O₃ 342.1943, found 342.1951.

### Example 18

### 2-((3R)-1-{[5-(2-fluorophenyl)isoxazol-4-yl]carbonyl}piperidin-3-yl)propan-2-ol

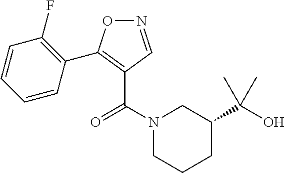

A solution of 5-(2-fluorophenyl)isoxazole-4-carboxylic acid (Intermediate 10; 81.6 mg, 0.394 mmol) and TBTU (132 mg, 0.411 mmol) in CH₂Cl₂ (6 mL) was added to a mixture of 2-[(3R)-piperidin-3-yl]propan-2-ol hydrochloride (Intermediate 18; 73.4 mg, 0.408 mmol) and triethylamine (0.1 mL, 0.719 mmol) in CH₂Cl₂ (2 mL). The mixture was stirred at room temperature for 30 min. The mixture was diluted with diethyl ether, washed with sat aq NaHCO₃ solution, 5% aq HOAc solution and then dried (MgSO₄). The solvent was evaporated and the residue was purified by flash chromatography (diethyl ether/petroleum ether/MeOH 70:25:5) to give 49 mg of the title compound.

HRMS (ESI+) calcd for C₁₈H₂₁FN₂O₃ 332.1536, found 332.1543.

### Example 19

### 2-((3R)-1-{[5-(4-fluorophenyl)isoxazol-4-yl]carbonyl}piperidin-3-yl)propan-2-ol

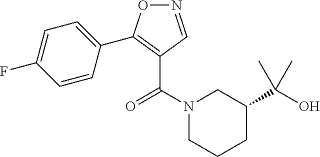

A solution of 5-(4-fluorophenyl)isoxazole-4-carboxylic acid (Intermediate 11; 116.0 mg, 0.560 mmol) and TBTU (195 mg, 0.607 mmol) in CH₂Cl₂ (6 mL) was added to a mixture of 2-[(3R)-piperidin-3-yl]propan-2-ol hydrochloride (Intermediate 18; 107.0 mg, 0.595 mmol) and triethylamine (0.083 mL, 0.600 mmol) in CH₂Cl₂ (2 mL). The mixture was stirred at room temperature for 1 h and then diluted with diethyl ether. The organic phase was washed with sat aq NaHCO₃ solution, 5% aq HOAc solution and then dried (MgSO₄). The solvent was evaporated and the residue was purified by flash chromatography (diethyl ether/petroleum ether/MeOH 70:25:5) to give 174 mg of the title compound.

HRMS (ESI+) calcd for C₁₈H₂₁FN₂O₃ 332.1536, found 332.1527.

### Example 20

### 2-((3S)-1-{[5-(4-fluorophenyl)isoxazol-4-yl]carbonyl}piperidin-3-yl)propan-2-ol

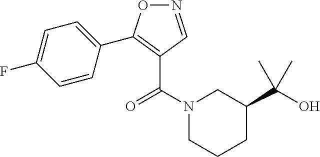

A solution of 5-(4-fluorophenyl)isoxazole-4-carboxylic acid (Intermediate 11; 52.0 mg, 0.301 mmol) and TBTU (101 mg, 0.315 mmol) in CH₂Cl₂ (2 mL) was added to a mixture of 2-[(3S)-piperidin-3-yl]propan-2-ol hydrochloride (Intermediate 17; 54.1 mg, 0.301 mmol) and triethylamine (0.048 mL, 0.3 mmol) in CH₂Cl₂ (2 mL). The mixture was stirred at room temperature for 1 hr. The crude product was purified by preparative HPLC (Xterra C18, 10 mM NH₄HCO₃ (pH 10)-CH₃CN) (5-30% MeCN) to give 24 mg of the title compound.

HRMS (ESI+) calcd for C₁₈H₂₁FN₂O₃ 332.1536, found 332.1534.

### Example 21

### 2-((3R)-1-{[5-(1-naphthyl)isoxazol-4-yl]carbonyl}piperidin-3-yl)propan-2-ol

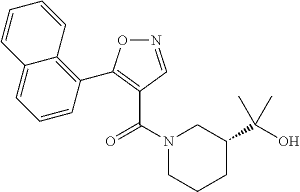

A solution of 5-(1-naphthyl)isoxazole-4-carboxylic acid (Intermediate 12; 101 mg, 0.424 mmol) and TBTU (140 mg, 0.436 mmol in CH₂Cl₂ (6 mL) was added to a mixture of 2-[(3R)-piperidin-3-yl]propan-2-ol hydrochloride (Intermediate 18; 80.1 mg, 0.446 mmol) and triethylamine (118 μL, 0.85 mmol) in CH₂Cl₂ (2 mL). The mixture was stirred at room temperature for 30 min and then diluted with diethyl ether. The organic phase was washed with sat aq NaHCO₃ solution, 5% aq HOAc solution and dried (MgSO₄). The solvent was evaporated and the residue was purified by flash chromatography (diethyl ether/petroleum ether/MeOH 70:25:5) to give 44 mg of the title compound.

HRMS (ESI+) calcd for C₂₂H₂₄N₂O₃ 364.1787, found 364.1781.

### Example 22

### 2-((3S)-1-{[3-(2-methylphenyl)isoxazol-4-yl]carbonyl}piperidin-3-yl)propan-2-ol

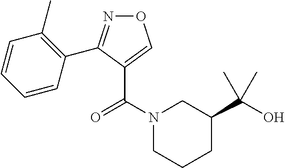

3-(2-methylphenyl)isoxazole-4-carboxylic acid (Intermediate 14; 137 mg, 0.674 mmol) and TBTU (221 mg, 0.688 mmol), dissolved in CH₂Cl₂ (4 mL), was added to a solution of 2-[(3S)-piperidin-3-yl]propan-2-ol hydrochloride (Intermediate 17; 121 mg, 0.673 mmol) and NEt₃ (189 μL, 1.35 mmol) in CH₂Cl₂ (4 mL). The mixture was stirred at 22° C. for 90 min, concentrated and purified by flash chromatography (petroleum ether/EtOAc/MeOH gradient 6:4:0 →48:48:4) to furnish 47 mg of the title compound as a grey solid.

HRMS (ESI+) calcd for C₁₉H₂₄N₂O₃ 328.1787, found 328.1788.

### Example 23

### 2-((3R)-1-{[3-(2-methylphenyl)isoxazol-4-yl]carbonyl}piperidin-3-yl)propan-2-ol

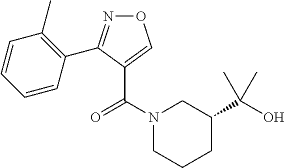

3-(2-Methylphenyl)isoxazole-4-carboxylic acid (Intermediate 14; 92.0 mg, 0.452 mmol) and TBTU (145 mg, 0.452 mmol) dissolved in CH₂Cl₂ (5 mL) was added to a solution of 2-[(3R)-piperidin-3-yl]propan-2-ol hydrochloride (Intermediate 18; 97.5 mg, 0.545 mmol) and NEt₃ (126 μL, 0.935 mmol) in CH₂Cl₂ (4 mL). The mixture was stirred at 22° C. for 90 min, concentrated and purified by flash chromatography (petroleum ether/EtOAc/MeOH gradient 6:4:0 →48:48:4) to furnish 81 mg of the title compound as a white foam.

HRMS (ESI+) calcd for C₁₉H₂₄N₂O₃ 328.1787, found 328.1786.

### Example 24

### 2-((3R)-1-{[3-(2-chlorophenyl)isoxazol-4-yl]carbonyl}piperidin-3-yl)propan-2-ol

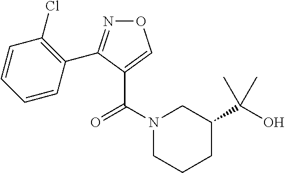

3-(2-chlorophenyl)isoxazole-4-carboxylic acid (Intermediate 15; 795 mg, 3.56 mmol) dissolved in SOCl₂ (10 mL) was heated to 110° C. for 20 min in a microwave reactor. The mixture was concentrated in vacuo, dissolved in CH₂Cl₂ (5 mL) and added to a solution of 2-[(3R)-piperidin-3-yl]propan-2-ol hydrochloride (Intermediate 18; 630 mg, 3.51 mmol) and NEt₃ (1.24 mL, 8.90 mmol) in CH₂Cl₂ (20 mL) at room temperature. The reaction mixture was stirred for 30 min and diluted with CH₂Cl₂ (20 mL). The organic phase was washed with 5% aq HOAc solution, brine and dried (MgSO₄). Evaporation of the solvent and triturating the crude in toluene furnished 977 mg of the title compound as a white solid.

HRMS (ESI+) calcd for C₁₈H₂₁ClN₂O₃ 348.1241, found 348.1240.

### Example 25

### 2-((3S)-1-{[3-(2-chlorophenyl)isoxazol-4-yl]carbonyl}piperidin-3-yl)propan-2-ol

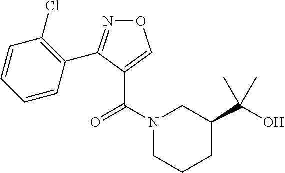

3-(2-chlorophenyl)isoxazole-4-carboxylic acid (Intermediate 15; 1.00 g, 4.48 mmol) dissolved in SOCl₂ (10 mL) was heated to 110° C. for 20 min in a microwave reactor. The mixture was concentrated in vacuo, dissolved in CH₂Cl₂ (5 mL) and added to a solution of 2-[(3S)-piperidin-3-yl]propan-2-ol hydrochloride (Intermediate 17; 807 mg, 4.49 mmol) and NEt₃ (1.56 mL, 11.2 mmol) in CH₂Cl₂ (20 mL) at room temperature. The reaction mixture was stirred for 30 min. The mixture was diluted with CH₂Cl₂ (20 mL), and washed with 5% aq HOAc solution, brine, and then dried (MgSO₄). Evaporation of the solvent and triturating the crude in toluene furnished 1.32 g of the title compound as a white solid.

HRMS (ESI+) calcd for C₁₈H₂₁ClN₂O₃ 348.1241, found 348.1242.

### Example 26

### 1-(1-{[3-(2-chlorophenyl)isoxazol-4-yl]carbonyl}piperidin-3-yl)ethanol

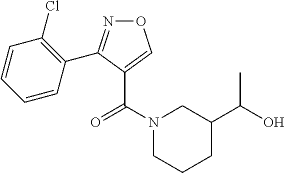

3-(2-chlorophenyl)isoxazole-4-carboxylic acid (Intermediate 15; 149 mg, 0.666 mmol) dissolved in SOCl₂ (3 mL) was heated at 120° C. for 15 min in a microwave reactor. The mixture was concentrated in vacuo, dissolved in CH₂Cl₂ (5 mL) and added to a solution of 1-(piperidin-3-yl)etan-1-ol (Intermediate 20; 130 mg, 0.785 mmol) and NEt₃ (0.228 mL, 1.64 mmol) in CH₂Cl₂ (4 mL) at room temperature. The reaction mixture was stirred for 30 min. The mixture was diluted with CH₂Cl₂ (20 mL), washed with 1M aq HCl solution and brine, and then dried (MgSO₄). The mixture was purified by flash chromatography (CH₂Cl₂/MeOH gradient 98:2 →94:6) to give 99.1 mg of a clear oil of the title compound as a mixture of 4 diastereomers.

HRMS (ESI+) calcd for C₁₇H₁₉ClN₂O₃ 334.1084, found 334.1085.

### Example 27

### 2-((3R)-1-{[3-(2,4-dimethylphenyl)isoxazol-4-yl]carbonyl}piperidin-3-yl)propan-2-ol

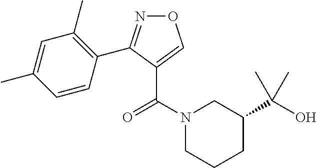

3-(2,4-Dimethylphenyl)isoxazole-4-carboxylic acid (Intermediate 13; 135 mg, 0.621 mmol) dissolved in SOCl₂ (2 mL) was heated at 110° C. for 30 min in a microwave reactor. The mixture was concentrated in vacuo, dissolved in CH₂Cl₂ (5 mL) and added to a solution of 2-[(3R)-piperidin-3-yl]propan-2-ol hydrochloride (Intermediate 18; 112 mg, 0.623 mmol) and NEt₃ (0.173 mL, 1.24 mmol) in CH₂Cl₂ (4 mL) at room temperature. The reaction mixture was stirred for 30 min and then diluted with diethyl ether. The organic phase was washed with 1M aq HCl solution, brine, and then dried (MgSO₄). The residue was crystallized from diethyl ether/petroleum ether to give 152 mg of the title compound as a white solid.

HRMS (ESI+) calcd for C₂₀H₂₆N₂O₃ 342.1943, found 342.1947.

### Example 28

### 2-((3S)-1-{[3-(2,4-dimethylphenyl)isoxazol-4-yl]carbonyl}piperidin-3-yl)propan-2-ol

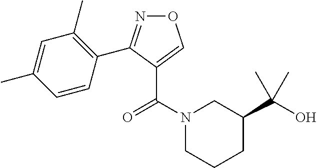

3-(2,4-dimethylphenyl)isoxazole-4-carboxylic acid (Intermediate 13; 115 mg, 0.529 mmol) dissolved in SOCl₂ (2 mL) was heated at 110° C. for 20 min in a microwave reactor. The mixture was concentrated in vacuo, dissolved in CH₂Cl₂ (5 mL) and added to a solution of 2-[(3S)-piperidin-3-yl]propan-2-ol hydrochloride (Intermediate 17; 105 mg, 0.584 mmol) and triethylamine (0.173 mL, 1.24 mmol) in CH₂Cl₂ (4 mL) at room temperature. The reaction mixture was stirred for 30 min and then diluted with diethyl ether. The organic phase was washed with 1M aq HCl solution, brine, and then dried (MgSO₄). The residue was crystallized from diethyl ether/petroleum ether to give 111 mg of the title compound as a beige solid.

HRMS (ESI+) calcd for C₂₀H₂₆N₂O₃ 342.1943, found 342.1949.

### Biological Methods

### Scintillation Proximity Assay

(1,2(n)-³H)-cortisone was purchased from Amersham Pharmacia Biotech. Anticortisol monoclonal mouse antibody, clone 6D6.7 was obtained from Immunotech and Scintillation proximity assay (SPA) beads coated with monoclonal antimouse antibodies were purchased from Amersham Pharmacia Biotech. NADPH tetrasodium salt was from Calbiochem and glucose-6-phosphate (G-6-P) was supplied by Sigma. The human 11β-hydroxysteroid dehydrogenase type-1 enzyme (11β-HSD 1) was expressed in Pichia pastoris. 18-β-glycyrrhetinic acid (GA) was obtained from Sigma. The serial dilutions of the compounds were dissolved in DMSO (1 mM) and diluted in 50 mM Tris-HCl, pH 7.2 containing 1 mM EDTA. The multiplication of plates was done on a Wallac Quadra. The amount of the product [³H]-cortisol, bound to the beads was determined in a Packard, Top Count microplate liquid scintillation counter.

The 11β-HSD1 enzyme assay was carried out in 96 well microtiter plates (Packard Optiplate) in a total well volume of 220 μL and contained 30 mM Tris-HCl, pH 7.2 with 1 mM EDTA, a substrate mixture tritiated Cortisone/NADPH (175 nM/181 μM), G-6-P (1 nM) and inhibitors in serial dilutions. Reactions were initiated by the addition of human 11β-HSD 1, either as Pichia pastoris cell homogenate or microsomes prepared from Pichia pastoris. Following mixing, the plates were shaken for 30 to 45 minutes at room temperature. The reactions were terminated with 10 μL 1 mM GA stop solution. Monoclonal mouse antibody was then added (10 μL of 4 μM) followed by 100 μL of SPA beads (suspended according to the manufacturers instructions). Appropriate controls were set up by omitting 11β-HSD1 to obtain the non-specific binding (NSB) value. The plates were covered with plastic film and incubated on a shaker for 30 minutes, at room temperature, before counting. The amount of [³H]-cortisol, bound to the beads was determined in a microplate liquid scintillation counter. The calculation of the Kᵢ values for the inhibitors was performed by use of Activity Base. The Kᵢ value is calculated from IC₅₀ and the Kₘ value is calculated using the Cheng Prushoff equation (with reversible inhibition that follows the Michaelis-Menten equation): Kᵢ=IC₅₀(1+[S]/Kₘ) [Cheng, Y. C.; Prushoff, W. H. Biochem. Pharmacol. 1973, 22, 3099-3108]. The IC₅₀ is measured experimentally in an assay wherein the decrease of the turnover of cortisone to cortisol is dependent on the inhibition potential of each substance.

The Kᵢ values of the compounds according to Examples 1-28 for 11β-HSD1 were typically between about 5 nM and about 600 nM.

### HTM-Cell Assay

MYOC gene product, myocilin, is expressed in many ocular tissues including the trabecular meshwork (TM) and ciliary body (structures of the eye involved in the regulation of intraocular pressure). Mutations of the MYOC gene have been associated with increased intraocular pressure and some forms of open angle glaucoma. The protein is induced to a high extent by glucocorticoids and is suggested to be involved in the regulation the aqueous humor outflow resistance (See e.g. Nguyen T. D. et al. (1998) J. Biol. Chem. 273, 6341-6350, Tamm E. R. (2002) Prog Retin Eye Res 21, 395-428). Furthermore, 11β-hydroxysteroid dehydrogenase type 1 (11β-HSD1) is the enzyme responsible for the intra-cellular conversion of the inactive glucocorticoid cortisone to the active steroid hormone cortisol.

Human trabecular meshwork (HTM) cell primary cultures were isolated from explants obtained from human eye banks and were used in two different types of experiments. For dose-response experiments, cells were incubated for 24 hours with a serial dilution of the test compounds (10 μM to 565 pM in cell medium containing 100 nM cortisone). Controls were medium only (negative control) and 100 nM cortisone (positive control). Cortisol levels in the harvested media were determined with a Cortisol-EIA Kit and from the resulting dose-response curves an IC₅₀ value of 3-30 nM was found.

Long-term experiments were performed to examine if the expression of the MYOC and 11β-HSD 1 genes could be correlated to cortisol levels. Experiments lasted for three weeks and started with a 7-day incubation period with cortisone, followed by 7-days incubation with compound in the presence of cortisone. To investigate if the effect of the compound lasts for one and up to three days, it was withdrawn from the medium and cortisone added solely. Culture medium was harvested for cortisol analysis and cell lysates were prepared for gene expression analysis on days 7, 14, 15 and 17. Controls in all steps were medium only (negative control) and 100 nM cortisone (positive control). Medium changes were made every third day. Cortisol levels in the harvested media were determined with the Cortisol-EIA Kit. RNA was extracted from cell lysates after the 17-days treatment and gene expression were measured by relative quantitative and real-time TaqMan polymerase chain reaction. As controls 18S RNA and β-actin were used. RNA levels were normalized to controls to avoid differences due to cell density.

Compared to positive controls, a 7-day treatment with inhibitor plus cortisone resulted in a prevention of cortisol production of 98%. Interestingly, one and three days after the withdrawal of the inhibitor, the cortisol production remains low and stays at about 20% and 50% of their respective controls. In a similar way, the MYOC and the 11β-HSD1 genes are affected by the inhibitor. A 2- to 10-fold up-regulation of the MYOC gene was found after 7 to 14 days, respectively, of cortisone treatment. On the other hand, the cells treated 7 days with cortisone alone and a further 7-day period with inhibitor plus cortisone stopped up-regulation of the gene and remained at about the same expression level as after the first week of cortisone treatment. One day after removal of the inhibitor the expression of MYOC is restored to about 40% of the control and three days after the up-regulation it reaches about 70% of the cortisone control. At the same conditions the 11β-HSD1 gene expression was increased 2- to 5-fold and withdrawal of the inhibitor gradually restores the up-regulation achieved by cortisone.

In summary, the broad influence of the compounds of the invention on the inhibition of cortisol production correlates with the effects on the expression of gene products connected with increased intraocular pressure and demonstrates its long lasting effect on the pharmacologically relevant trabecular meshwork cells.

### Cornea Permeability Screening Tool

A selected number of compounds as described herein were tested for their ocular penetration after topical administration in pig eyes in vitro as an aid in deciding their potential as ocular drug candidates. Fresh eyes were delivered from animals that had been sacrificed during the morning each day of the experimental period. The eyes were transferred to Falcon tubes 50 ml, containing +37° C. BSS in order to allow the corneal endothelium to become metabolically active. Before topical administration of the test substance, the eyes were placed in a moisture chamber in order to avoid excessive drying of the eyes during the longer incubations.

Pig eyes were exposed to a solution or suspension of the compound in phosphate buffer. Cremophor® RH40 (polyoxol castor oil, BASF) was routinely used as a formulation excipient. For each formulation, three incubation times were used. For each time-point, six eyes were used. Before administration the eyes were washed with BSS followed by the application of 50 μl of phosphate buffer, in order to moisten the corneal surface. A single drop (20 μl) of the formulation was applied at the start of the incubation. At the end of the incubation time, the cornea was perforated with a 30G cannula and the aqueous humor (AH) was withdrawn and transferred to plastic tubes. The aqueous humor samples were stored at −18° C. until analysis.

For the compounds tested, the AH concentration was found to be in the range of 0.001-6 μM after 10 min exposure. A ranking of the compounds, as a screening tool, was subsequently done using the ratio of the compound concentration in the AH_{20 min}/IC₅₀. The ratio was found to be in the range of 30-400 for the compounds tested.

Consequently, compounds as described according to the invention were shown to be capable of ocular penetration after topical administration to pig eyes in vitro.

1. A compound of formula (I), 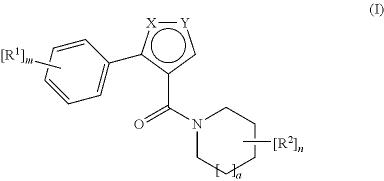
or a pharmaceutically acceptable salt, solvate, hydrate, geometrical isomer, tautomer, optical isomer or N-oxide thereof, wherein:
X—Y represents N—O or O—N;
R¹ is independently selected from the group consisting of halogen, cyano, CF₃, OCF₃, C₁₋₄-alkyl, hydroxy-C₁₋₄-alkyl, C₁₋₄-alkoxy-C₁₋₄-alkyl and C₁₋₄-alkoxy;
or two substituents R¹, together with the carbon atoms they are attached to, form a 5- or 6-membered aromatic or non-aromatic ring, which optionally contains one or more heteroatoms selected from O and N, and which ring is optionally substituted with one or more substituents selected from the group consisting of halogen, cyano, CF₃, OCF₃, C₁₋₄-alkyl, hydroxy-C₁₋₄-alkyl, C₁₋₄-alkoxy-C₁₋₄-alkyl and C₁₋₄-alkoxy;
R² is independently selected from the group consisting of C₁₋₈-alkyl, hydroxy-C₁₋₈-alkyl, C₁₋₈-alkoxy-C₁₋₈-alkyl, C₁₋₈-alkoxy, hydroxy-C₁₋₈-alkoxy, C₁₋₈-alkoxy-C₁₋₈-alkoxy, C₃₋₈-cycloalkyl, hydroxy-C₃₋₈-cycloalkyl, C₁₋₈-alkoxy-C₃₋₈-cycloalkyl, C₃₋₈-cycloalkyloxy, hydroxy-C₃₋₈-cycloalkyloxy and C₁₋₈-alkoxy-C₃₋₈-cycloalkyloxy;
a is 0, 1 or 2; and
m and n are each independently 0, 1 or 2;
with the proviso that the compound is not selected from the group consisting of:
1-[(5-phenyl-4-isoxazolyl)carbonyl]-piperidine;
5-phenyl-4-(pyrrolidin-1-ylcarbonyl)isoxazole;
5-(4-fluorophenyl)-4-(pyrrolidin-1-ylcarbonyl)isoxazole;
5-(4-chlorophenyl)-4-(pyrrolidin-1-ylcarbonyl)isoxazole;
5-(4-bromophenyl)-4-(pyrrolidin-1-ylcarbonyl)isoxazole;
5-(4-methylphenyl)-4-(pyrrolidin-1-ylcarbonyl)isoxazole;
5-(4-methoxyphenyl)-4-(pyrrolidin-1-ylcarbonyl)isoxazole;
4-{[2-(methoxymethyl)pyrrolidin-1-yl]carbonyl}-5-(4-methylphenyl)isoxazole;
4-{[(2S)-2-(methoxymethyl)pyrrolidin-1-yl]carbonyl}-5-(4-methylphenyl)-isoxazole;
4-{[(2R)-2-(methoxymethyl)pyrrolidin-1-yl]carbonyl}-5-(4-methylphenyl)-isoxazole;
5-(2-chlorophenyl)-4-{[2-(methoxymethyl)pyrrolidin-1-yl]carbonyl}isoxazole;
5-(2-chlorophenyl)-4-{[(2S)-2-(methoxymethyl)pyrrolidin-1-yl]carbonyl}-isoxazole;
5-(2-chlorophenyl)-4-{[(2R)-2-(methoxymethyl)pyrrolidin-1-yl]carbonyl}-isoxazole;
4-{[(2S)-2-(methoxymethyl)pyrrolidin-1-yl]carbonyl}-5-[4-(trifluoromethyl)-phenyl]isoxazole;
4-{[(2R)-2-(methoxymethyl)pyrrolidin-1-yl]carbonyl}-5-[4-(trifluoromethyl)-phenyl]isoxazole;
5-(4-chlorophenyl)-4-{[2-(isopropyl)pyrrolidin-1-yl]carbonyl}isoxazole;
2-(1-{[5-(4-methylphenyl)isoxazol-4-yl]carbonyl}piperidin-3-yl)propan-2-ol;
2-(1-{[5-(2-chlorophenyl)isoxazol-4-yl]carbonyl}piperidin-3-yl)propan-2-ol;
2-[1-({5-[4-(trifluoromethyl)phenyl]isoxazol-4-yl}carbonyl)piperidin-3-yl]-propan-2-ol;
2-((3R)-1-{[3-(4-methylphenyl)isoxazol-4-yl]carbonyl}piperidin-3-yl)propan-2-ol;
2-((3R)-1-{[5-(4-methylphenyl)isoxazol-4-yl]carbonyl}piperidin-3-yl)propan-2-ol;
2-((3R)-1-{[3-(4-methoxyphenyl)isoxazol-4-yl]carbonyl}piperidin-3-yl)propan-2-ol;
2-((3R)-1-{[3-(3-chloro-4-methoxyphenyl)isoxazol-4-yl]carbonyl}piperidin-3-yl)-propan-2-ol;
3-(1-{[5-(4-methylphenyl)isoxazol-4-yl]carbonyl}piperidin-3-yl)pentan-3-ol;
3-(1-{[3-(4-methylphenyl)isoxazol-4-yl]carbonyl}piperidin-3-yl)pentan-3-ol;
3,5-dimethyl-1-{[5-(4-methylphenyl)isoxazol-4-yl]carbonyl}piperidine;
1-{[5-(2-chlorophenyl)isoxazol-4-yl]carbonyl}-3,5-dimethylpiperidine;
3,5-dimethyl-1-({5-[4-(trifluoromethyl)phenyl]isoxazol-4-yl}carbonyl)piperidine;
5-ethyl-2-methyl-1-{[5-(4-methylphenyl)isoxazol-4-yl]carbonyl}piperidine;
1-{[5-(2-chlorophenyl)isoxazol-4-yl]carbonyl}-5-ethyl-2-methylpiperidine; and
5-ethyl-2-methyl-1-({5-[4-(trifluoromethyl)phenyl]isoxazol-4-yl}carbonyl)-piperidine.

2. A compound according to claim 1 wherein a is 1.

3. A compound according to claim 1 or 2 wherein R¹ is halogen or C₁₋₄-alkyl, or wherein two substituents R¹, together with the carbon atoms they are attached to, form a 5- or 6-membered ring.

4. A compound according to claim 1 or 2 wherein R¹ is F, Cl or methyl, or wherein two substituents R¹, together with the carbon atoms they are attached to, form a 6-membered aromatic ring.

5. A compound according to claim 1 or 2 wherein R² is hydroxy-C₁₋₈-alkyl or C₁₋₈-alkoxy-C₁₋₈-alkoxy.

6. A compound according to claim 1 or 2 wherein R² is 1-hydroxyethyl, 2-hydroxyethyl or 1-hydroxy-1-methylethyl.

7. A compound according to claim 1 which is selected from:
2-(1-{[5-(2-methylphenyl)isoxazol-4-yl]carbonyl}piperidin-3-yl)propan-2-ol;
2-((3R)-1-{[5-(2-methylphenyl)isoxazol-4-yl]carbonyl}piperidin-3-yl)propan-2-ol;
2-((3S)-1-{[5-(2-methylphenyl)isoxazol-4-yl]carbonyl}piperidin-3-yl)propan-2-ol;
2-(1-{[5-(3-methylphenyl)isoxazol-4-yl]carbonyl}piperidin-3-yl)propan-2-ol;
2-((3S)-1-{[5-(4-methylphenyl)isoxazol-4-yl]carbonyl}piperidin-3-yl)propan-2-ol;
2-((3R)-1-{[5-(2-chlorophenyl)isoxazol-4-yl]carbonyl}piperidin-3-yl)propan-2-ol;
2-((3S)-1-{[5-(2-chlorophenyl)isoxazol-4-yl]carbonyl}piperidin-3-yl)propan-2-ol;
2-((3S)-1-{[5-(3-chlorophenyl)isoxazol-4-yl]carbonyl}piperidin-3-yl)propan-2-ol;
2-((3R)-1-{[5-(4-chlorophenyl)isoxazol-4-yl]carbonyl}piperidin-3-yl)propan-2-ol;
2-((3S)-1-{[5-(4-chlorophenyl)isoxazol-4-yl]carbonyl}piperidin-3-yl)propan-2-ol;
2-(1-{[5-(2-chlorophenyl)isoxazol-4-yl]carbonyl}piperidin-2-yl)ethanol;
2-(1-{[5-(3-chloro-2-methylphenyl)isoxazol-4-yl]carbonyl}piperidin-3-yl)propan-2-ol;
2-(1-{[5-(3,4-dimethylphenyl)isoxazol-4-yl]carbonyl}piperidin-3-yl)propan-2-ol;
2-((3R)-1-{[5-(3,4-dimethylphenyl)isoxazol-4-yl]carbonyl}piperidin-3-yl)propan-2-ol;
2-((3S)-1-{[5-(3,4-dimethylphenyl)isoxazol-4-yl]carbonyl}piperidin-3-yl)propan-2-ol;
2-((3R)-1-{[5-(2,4-dimethylphenyl)isoxazol-4-yl]carbonyl}piperidin-3-yl)propan-2-ol;
2-((3S)-1-{[5-(2,4-dimethylphenyl)isoxazol-4-yl]carbonyl}piperidin-3-yl)propan-2-ol;
2-((3R)-1-{[5-(2-fluorophenyl)isoxazol-4-yl]carbonyl}piperidin-3-yl)propan-2-ol;
2-((3R)-1-{[5-(4-fluorophenyl)isoxazol-4-yl]carbonyl}piperidin-3-yl)propan-2-ol;
2-((3S)-1-{[5-(4-fluorophenyl)isoxazol-4-yl]carbonyl}piperidin-3-yl)propan-2-ol;
2-((3R)-1-{[5-(1-naphthyl)isoxazol-4-yl]carbonyl}piperidin-3-yl)propan-2-ol;
2-((3S)-1-{[3-(2-methylphenyl)isoxazol-4-yl]carbonyl}piperidin-3-yl)propan-2-ol;
2-((3R)-1-{[3-(2-methylphenyl)isoxazol-4-yl]carbonyl}piperidin-3-yl)propan-2-ol;
2-((3R)-1-{[3-(2-chlorophenyl)isoxazol-4-yl]carbonyl}piperidin-3-yl)propan-2-ol;
2-((3S)-1′-{[3-(2-chlorophenyl)isoxazol-4-yl]carbonyl}piperidin-3-yl)propan-2-ol;
2-((3R)-1-{[3-(2,4-dimethylphenyl)isoxazol-4-yl]carbonyl}piperidin-3-yl)propan-2-ol;
1-(1-{[3-(2-chlorophenyl)isoxazol-4-yl]carbonyl}piperidin-3-yl)ethanol; and
2-((3S)-1-{[3-(2,4-dimethylphenyl)isoxazol-4-yl]carbonyl}piperidin-3-yl)propan-2-ol.

8. A pharmaceutical composition comprising a compound according to claim 1 as an active ingredient, and a pharmaceutically acceptable carrier, diluent or excipient.

9. The composition according to claim 8 comprising a compound according to claim 7.

10. A method for the treatment or prevention of glaucoma, comprising administering to a human subject in need of such treatment an effective amount of a compound according to claim 1.

11. The method according to claim 10, wherein the compound is a compound according to claim 7.
